(19) United States
(12) Patent Application Publication
(10) Pub. No.: US 2024/0140758 A1
(43) Pub. Date: May 2, 2024

(54) ELEVATOR OPERATION SYSTEM

(71) Applicant: Woon Hak IM, Seoul (KR)

(72) Inventor: Woon Hak IM, Seoul (KR)

(21) Appl. No.: 18/061,187

(22) Filed: Dec. 2, 2022

Related U.S. Application Data
(63) Continuation of application No. PCT/KR2020/018166, filed on Dec. 11, 2020.

(30) Foreign Application Priority Data
Jun. 5, 2020 (KR) 10-2020-0068414

(51) Int. Cl. B66B 1/46; A61L 2/10

(52) U.S. Cl. CPC B66B 1/461; A61L 2/10; A61L 2202/14

(57) ABSTRACT

Provided is an elevator operation system according to the present invention. The elevator operation system may comprise: an external operation panel configured to control, from the outside of the elevator, stopping of the elevator and opening and closing of the door of the elevator; and a control unit configured to be operable together with the external operation panel and to control sensors and LEDs associated with an up button and a down button on the basis of a user input on the up button and the down button of the external operation panel, thereby sterilizing and disinfecting a corresponding button whenever the user touches the elevator button.

### CROSS-REFERENCE TO RELATED APPLICATIONS

The present application is a continuation of International Patent Application No, PCT/KR2020/018166, filed on Dec. 11, 2020, which is based upon and claims the benefit of priority to Korean Patent Application No. 10-2020-0068414 filed on Jun. 5, 2020. The disclosures of the above-listed applications are hereby incorporated by reference herein in their entirety.

### TECHNICAL FIELD

The present invention relates to an elevator operation method and an elevator operation system, and more particularly, to an operation method of an elevator including an external operation panel and an internal operation panel and an elevator operation system for performing the same.

### RELATED ART

As a plurality of unspecific users touches a button of an operation panel outside and inside an elevator, infection by germs or pathogens may occur. In this regard, a prevention method using disinfectants and hand sanitizers may be considered as a countermeasure against viral pathogens such as corona, swine flu, and MFRS.

The prevention method using disinfectants and hand sanitizers may be performed while the elevator is not in use. However, when the elevator is in use, it is inconvenient for a manager other than a general user to perform such prevention method.

Meanwhile, as a method of disinfecting an elevator button, a method of spraying a disinfectant or separately attaching an antibacterial filter may be considered. However, although the antibacterial filter is separately attached, the effect may significantly decrease when a user presses a button a certain number of times or lore, or when a certain period of time elapses. Also, in the case of spraying a disinfectant and the like over an elevator button, the disinfectant may penetrate into the elevator button and damage a button switch. Also, there is an issue that disinfection may not be performed every time more than hundreds of persons continuously press a button switch a day.

### DETAILED DESCRIPTION

### Technical Object

Therefore, to solve the aforementioned issues, an object of the present invention is to provide an elevator operation system that may sterilize and disinfect a corresponding button every time a user touches an elevator button.

Also, an object of the present invention is to provide a button disinfection method that may sterilize and disinfect a corresponding button every time a user touches an elevator button and an elevator operation system for performing the same.

### Solution

To accomplish the aforementioned objects, an elevator operation system according to the present invention is provided. The elevator operation system includes an external operation panel configured to control stopping of an elevator and opening and closing of a door of the elevator from the outside of the elevator; and a controller configured to be operable with the external operation panel and to control a sensor and a light emitting diode (LED) associated with an up button and a down button of the external operation panel based on a user input on the up button and the down button of the external operation panel, and thereby may sterilize and disinfect a corresponding button every time a user touches an elevator button.

In an embodiment, the up button and the down button may be provided in an internal area at a predetermined interval from the plane of the external operation panel.

In an embodiment, the controller may recognize that the up button is pressed by emitting infrared (IR) rays from a first transmission sensor according to a touch motion around the up button and by receiving the IR rays at a first reception sensor. When the up button is recognized as being pressed, the controller may drive a first ultraviolet (UV) LED and control the first UV LED such that UV sterilization and disinfection is performed in an area around the up button.

In an embodiment, the controller may recognize that the down button is pressed by emitting IR rays from a second transmission sensor according to a touch motion around the down button and by receiving the IR rays at a second reception sensor. When the down button is recognized as being pressed, the controller may drive a second UV LED and control the second UV LED such that UV sterilization and disinfection is performed in an area around the down button.

In an embodiment, when the up button is recognized as being pressed again during the UV sterilization and disinfection by the first UV LED, the controller may suspend driving of the first UV LED. When the down button is recognized as being pressed again during the UV sterilization and disinfection by the second UV LED, the controller may suspend driving of the second UV LED.

In an embodiment, the controller may recognize that the down button is pressed by emitting IR rays from a second transmission sensor according to a touch motion around the down button during the UV sterilization and disinfection in the area around the up button, and by receiving the IR rays at a second reception sensor. The controller may suspend driving of the first UV LED according to a touch motion around the down button, drive a second UV LED, and control the second UV LED such that UV sterilization and disinfection is performed in an area around the down button.

In an embodiment, the elevator operation system may further include an internal operation panel configured to control a floor on which the elevator arrives and stops and opening and closing of the door of the elevator inside the elevator, The controller may be configured to be operable with the internal operation panel and control a sensor and an LED associated with a corresponding button based on a user input on an open button, a close button, and a plurality of buttons corresponding to the floor to be stopped of the internal operation panel.

In an embodiment, the open button, the close button, and the plurality of buttons corresponding to the floor to be stopped of the internal operation panel may be arranged in an internal area at a predetermined interval from the plane of the internal operation panel. The controller may recognize that a first button is pressed by emitting IR rays from a first transmission sensor closest to the first button according to a touch motion around the first button among the open button, the close button, and the plurality of buttons corresponding to the floor to be stopped of the internal operation panel and by receiving the IR rays at the first reception sensor. When the first button is recognized as being pressed, the controller may drive a first UV LED and control the first UV LED such that UV sterilization and disinfection is performed in an area around the first button.

In an embodiment, when the first button is recognized as being pressed again during the UV sterilization and disinfection by the first UV LED, the controller may suspend driving of the first UV LED.

In an embodiment, the controller may recognize that a second button is pressed by emitting IR rays from a second transmission sensor closest to a second button according to a touch motion around the second button among the plurality of buttons of the internal operation panel and by receiving the IR rays at the second reception sensor. When the second button is recognized as being pressed again, the controller may suspend driving of the first UV LED, drive a second UV LED, and control the second UV LED such that UV sterilization and disinfection is performed in an area around the second button.

In an embodiment, when the second button is recognized as being pressed again during the UV sterilization and disinfection by the second UV LED, the controller may suspend driving of the second UV LED.

In an embodiment, if light intensity of the first UV LED is less than or equal to a threshold during the UV sterilization and disinfection by the first UV LED, the controller nay drive a third UV LED closest to the first UV LED and control UV sterilization and disinfection to be performed in the area around the first button.

In an embodiment, if light intensity of the first UV LED is less than or equal to a threshold during the UV sterilization and disinfection by the first UV LED and a touch motion is received around a fourth button within a predetermined distance from the first button, the controller may drive a fourth UV LED corresponding to the fourth button and control UV sterilization and disinfection to be performed in the area around the first button.

### Effect

According to the present invention, it is possible to provide an elevator operation system that may sterilize and disinfect a corresponding button every time a user touches an elevator button.

Also, according to the present invention, it is possible to provide a button disinfection method that may sterilize and disinfect a corresponding button every time a user touches an elevator button and an elevator operation system for performing the same.

Also, according to the present invention, it is possible to provide a button disinfection method that may sterilize and disinfect a corresponding button by reflecting the intent of a user every time each button provided to an external operation panel of an elevator is touched and an elevator operation system for performing the same.

Also, according to the present invention, it is possible to provide a button disinfection method that may sterilize and disinfect a corresponding button by reflecting the intent of a user every time each button provided to an internal operation panel of an elevator is touched and an elevator operation system for performing the same.

Also, according to the present invention, it is possible to provide a button disinfection method that may sterilize and disinfect a corresponding button in consideration of the intent of a user and a sterilization and disinfection capability associated with each button every time each button provided to an internal operation panel or an external operation panel of an elevator is touched and an elevator operation system for performing the same.

The features and effects of the present invention may become more apparent through the following detailed description in relation to the accompanying drawings and accordingly, those skilled in the art to which the present invention pertains may easily carry out the technical spirit of the present invention.

### BRIEF DESCRIPTION OF DRAWINGS

FIG. 1 illustrates a modified example of an external operation panel of an elevator according to the present invention.

FIG. 2 illustrates a modified example of an internal operation panel of an elevator according to the present invention.

FIG. 3 illustrates a configuration coupled to a sensor and a light emitting diode (LED) configured to operably couple to the external operation panel of FIG. 1.

FIG. 4 is a diagram illustrating a detailed configuration of an elevator operation system according to an embodiment of the present invention.

FIG. 5 is a diagram illustrating a detailed configuration of an elevator operation system according to another embodiment of the present invention.

FIG. 6 illustrates an external operation panel for controlling a common front door according to another embodiment of the present invention.

### MODE

The features and effects of the present invention may become more apparent through the following detailed description in relation to the accompanying drawings and accordingly, those skilled in the art to which the present invention pertains may easily carry out the technical spirit of the present invention.

Since various modifications may be made to the present invention and various embodiments may be implemented, specific embodiments will be illustrated in the drawings and described in detail the detailed description. However, the present invention is not construed as being limited to specific implementation and it should be understood to include all modification, equivalents, and substitutes included in the spirit and technical scope of the present invention.

In describing each drawing, like reference numerals refer to like elements.

Although terms, “first”, “second”, etc., may be used to explain various components, the components are not limited to the terms. The terms are used to distinguish one component from another component.

For example, a first component may be referred to as a second component, or similarly, the second component may be referred to as the first component within the scope of the right according to the concept of the present invention. Term “and/or” includes a combination of a plurality of related listed items or one of the listed items.

Unless otherwise defined herein, all terms used herein including technical or scientific terms have the same meanings as those generally understood by one of ordinary skill in the art.

Terms defined in dictionaries generally used should be construed to have meanings matching contextual meanings in the related art and are not to be construed as an ideal or excessively formal meaning unless otherwise defined herein.

The suffixes, “˜module”, “˜block”, and “˜unit” for components used in the following description are assigned or used interchangeably in consideration of ease of writing the specification only and do not have meanings or roles that are distinguished from each other by themselves.

Hereinafter, embodiments of the present invention will be described such that one of ordinary skill in the art may easily implement the same with reference to the accompanying drawings. In describing the embodiments of the present invention below, when it is determined that detailed description related to a known function or a known configuration makes the subject matter of the present invention unnecessarily ambiguous, the detailed description is omitted.

Hereinafter, an elevator operation method and an elevator operation system for performing the same according to the present invention will be described.

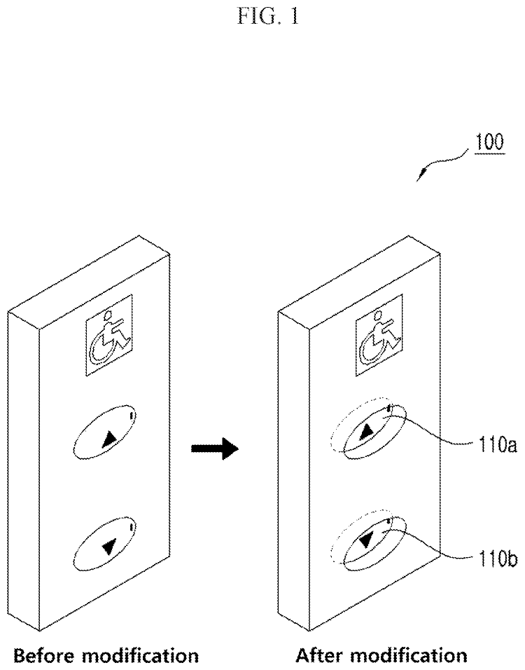

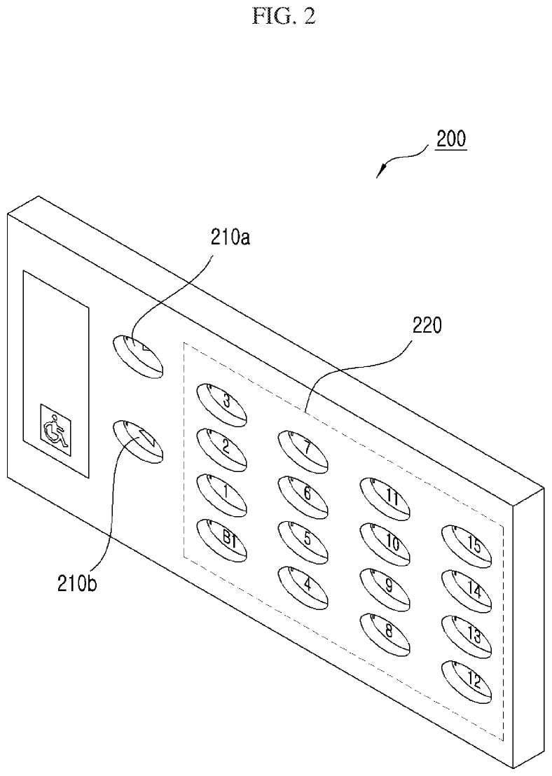

In this regard, FIG. 1 illustrates a modified example of an external operation panel of an elevator according to the present invention. FIG. 2 illustrates a modified example of an internal operation panel of an elevator according to the present invention. Referring to FIG. 1, an up button 110a and a down button 110b of an external operation panel 100 may be arranged in an internal area at a predetermined interval based on a body of the external operation panel 100. The up button 110a and the down button 110b may be configured to use the external operation panel MO before modification. The up button 110a and the down button 110b may be provided in the internal area of the external operation panel 100 as injection molding objects with the same size.

Also, referring to FIG. 2, an open button 210a, a close button 210b, and a plurality of buttons 220 corresponding to a floor to be stopped of an internal operation panel 200 may be provided in an internal area at a predetermined interval based on a body of the internal operation panel 200. The open button 210a, the close button 210h, and the plurality of buttons 220 may be configured to use the internal operation panel 200 before modification. The open button 210a, the close button 210b, and the plurality of buttons 220 may be provided in the internal area of the internal operation panel 200 as injection molding objects with the same size.

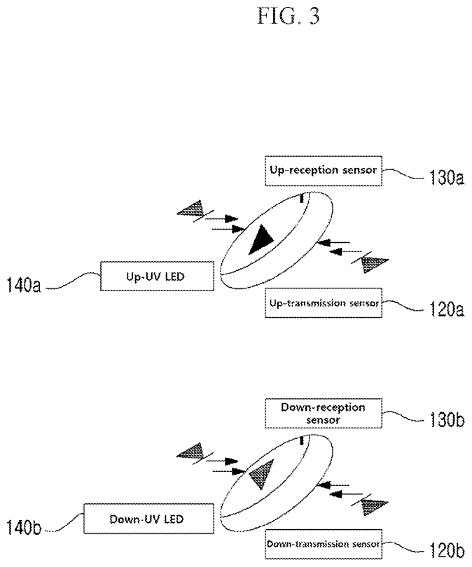

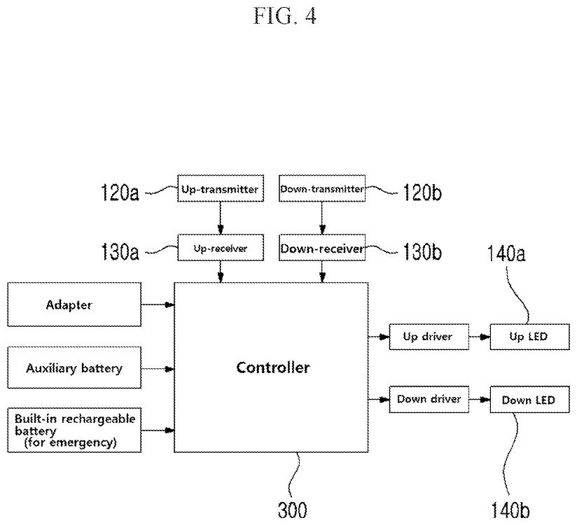

FIG. 3 illustrates a configuration coupled to a sensor and a light emitting diode (LED) configured to operably couple external operation panel of FIG. 1. FIG. 4 is a diagram illustrating a detailed configuration of an elevator operation system according to an embodiment of the present invention.

According to the present invention, an elevator may be configured to ascend or descend to a basement, a first floor, and each floor of a building. To this end, an operation panel configured to control an operation of the elevator may include a plurality of buttons. Meanwhile, equipment specially manufactured to enable sterilization and disinfection for each button may be provided to a button for controlling the elevator to ascend or descend to each floor and buttons corresponding to the respective floors. Also, even through the separately manufactured equipment, it is necessary to enable the sterilization and disinfection for each button while recognizing a button pressing motion according to a touch motion of a user. To this end, the present invention provides a device for disinfecting an N^{th} floor button including up/down buttons and basement floors with ultraviolet (UV) rays (a UV lamp or a UV LED). Meanwhile, if separate equipment with a height of several trim may not be mounted, an internal/external operation panel configured to control the elevator by mounting a separate jig may be configured.

Referring to FIGS. 1 to 4, the elevator operation system according to the present invention may include the external operation panel 100 and a controller 300. Meanwhile, the elevator operation system may include the internal operation panel 200 and the controller 300. Alternatively, the elevator operation system may include the external operation panel 100, the internal operation panel 200, and the controller 300.

Regarding a detailed operation of the present invention, when the user presses an up button, a microcomputer may recognize whether the up button is pressed by emitting infrared (IR) rays from an up-transmission sensor and by receiving the IR rays at a reception sensor. Accordingly, the controller 300 corresponding to the microcomputer may drive an up-button UV LED for 0-1 minutes (time adjustable) and may control the up-button UV LED to disinfect a corresponding button. Also, when the up button is pressed during the disinfection, the controller 300 may immediately suspend driving of the up-button UV LED. For example, in the present invention, when the user presses the up button, the controller 300 may disinfect the up button and may determine tether a specific amount of time (e.g., 0-1 minutes) elapses during disinfection. Unless the specific amount of time elapses during the disinfection, the microcomputer may count a remaining time and may need to disinfect a corresponding button for the specific amount of time or more e.g., 1 minute or longer). That is, the present invention may recognize a pressed button among a plurality of buttons and ay immediately disinfect the corresponding button.

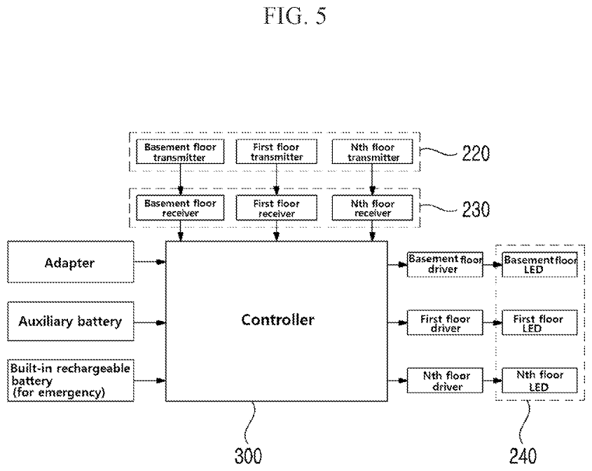

Referring to FIGS. 4 and 5, when external power (adapter) is unavailable according to the Fire Protection Act, an auxiliary battery may be installed and used for power to be used inside and outside the elevator. Also, a built-in rechargeable battery may be installed and used for the power to be used inside and outside the elevator. In this regard, FIG. 5 illustrates a detailed configuration of an elevator operation system according to another embodiment of the present invention. Here, the elevator operation system may operably couple to the internal operation panel.

Referring to FIGS. 1, 3, and 4, in a type A configuration, that is, the elevator operation system that controls the external operation panel, the controller 300 includes a first transmission sensor 120a and a first reception sensor 130a corresponding to an up-transmitter and an up-receiver, respectively. Also, the elevator operation system further includes a second transmission sensor 120b and a second reception sensor 130b corresponding to a down-transmitter and a down receiver, respectively. Also, the elevator operation system may further include a first UV LED 140a and a second UV LED 140b corresponding to an up-UV LED and a down-UV LED, respectively. In this regard, the elevator operation system may further include an up driver and a down driver configured to operably couple to the first UV LED 140a and the second UV LED 140b, respectively. Also, the elevator operation system may further include an external power (adaptor), an auxiliary battery, and a built-in rechargeable battery in relation to a power supplier.

Referring to FIGS. 2 and 5, a type B configuration, that is, the elevator operation system that controls the internal operation panel may include a plurality of transmitters 220 that interacts with the plurality of buttons 220 corresponding to the respective floors, a plurality of receivers 230, and a plurality of LEDs 240. The plurality of transmitters 220 may include a basement floor transmitter, and a first floor transmitter to an N^{th} floor transmitter. The plurality of receivers 230 may include a basement floor receiver and a first floor receiver to an N^{th} floor receiver. The plurality of LEDs 240 may include a basement floor LED and a first floor LED to an N^{th} floor LED.

For example, the elevator operation system may include the basement floor transmitter and the basement floor receiver. Also, the elevator operation system may include the first floor transmitter and the first floor receiver. Also, the elevator operation system may include the N^{th} floor transmitter and the N^{th} floor receiver. In this regard, a transmitter and a receiver corresponding to one button among a plurality of buttons may be referred to as a first transmission sensor and a second reception sensor, respectively. A transmitter and a receiver corresponding to another button among the plurality of buttons may be referred to as a second transmission sensor and a second reception sensor.

For example, the elevator operation system may include a basement floor driver and a basement floor LED. Also, the elevator operation system may include a first floor driver and the first floor LED. Also, the elevator operation system may include an N^{th} floor driver and an N^{th} floor LED. In this regard, a UV LED corresponding to one button among the plurality of buttons may be referred to as a first UV LED. Also, a UV LED corresponding to another button among the plurality of buttons may be referred to as a second UV LED.

The external operation panel 100 according to the present invention may be configured to control stopping of the elevator and opening and closing of the door of the elevator from the outside of the elevator. The controller 300 may be configured to be operable with the external operation panel 100. The controller 300 may be configured to control a sensor and an LED associated with the up button 110a and the down button 110b based on a user input on the up button 110a and the down button 110b of the external operation panel 100.

Referring to FIG. 1, the up button 110a and the down button 110b may be provided in the internal area at the predetermined interval from the plane of the external operation panel 100. The controller 300 may control the first transmission sensor 120a to emit IR rays according to a touch motion around the up button 110a. Also, the controller 300 may recognize that the up button 110a is pressed by receiving the IR rays at the first reception sensor 130a. When the up button 110a is recognized as being pressed, the controller 300 may drive the first UV LED 140a and may control the first UV LED 140a such that UV sterilization and disinfection is performed in an area around the up button 110a.

The controller 300 may individually recognize a button touch even for the down button 110b and may individually control a UV LED such that UV sterilization and disinfection is performed for each button. Therefore, the present invention may perform sterilization and disinfection on a corresponding button every time the corresponding button is touched. In this regard, the controller 300 may control the second transmission sensor 120b to emit IR rays according to a touch motion around the down button 110b. Also, the controller 300 may recognize that the down button 110b is pressed by receiving the IR rays at the second reception sensor 130b. When the down button 110b is recognized as being pressed, the controller 300 may drive the second UV LED 140b and may control the second UV LED 140b such that UV sterilization and disinfection is performed in an area around the down button 110b.

Referring to FIG. 3, the first transmission sensor 120a and the first reception sensor 130a may correspond to an up-transmission sensor and an up-reception sensor, respectively. Also, the first UV LED 140a may correspond to an up-UV LED. Also, the second transmission sensor 120b and the second reception sensor 130h may correspond to a down-transmission sensor and a down-reception sensor, respectively. Also, the second UV LED 140h may correspond to a down-UV LED.

Therefore, the controller 300 may individually determine whether the up button 110a and the down button 110b of the external operation panel 100 are pressed and may control sterilization and disinfection to be performed on each button. Referring to FIG. 2, the controller 300 may individually determine whether the open button 210a, the close button 210b, and the plurality of buttons 220 corresponding to a floor to be stopped of the internal operation panel 200 are pressed and may control sterilization and disinfection to be performed on each button.

According to an embodiment of the present invention, when a user touches again a corresponding button, it is possible to suspend disinfection and sterilization operation on the corresponding button by reflecting the intent of the user. In this regard, if a predetermined period of time elapses after the disinfection and sterilization operation is initiated by the user touching the corresponding button, the disinfection and sterilization operation on the corresponding button may be completed. However, when the user determines that the disinfection and sterilization operation is sufficiently performed on the corresponding button and touches gain the corresponding button, this intent may be reflected and the disinfection and sterilization operation on the corresponding button may be suspended.

In this regard, when the up button 110a is recognized as being pressed again during the UV sterilization and disinfection by the first UV LED 140a, the controller 300 may suspend driving of the first UV LED 140a. When the down button 110b is recognized as being pressed again during the UV sterilization and disinfection by the second UV LED 140b, the controller 300 may suspend driving of the second. UV LED 140b.

According to another embodiment of the present invention, when a touch motion another button is recognized during disinfection and sterilization operation being performed on one button between the up button and the down button, it is possible to control a disinfection and sterilization operation by reflecting the intent of the user. For example, the controller 300 may control the second transmission sensor 120b to emit IR rays according to a touch motion around the down button 110b during the UV sterilization and disinfection in the area around the up button 110a. The controller 300 may recognize that the down button 110b is pressed by receiving the IR rays at the second reception sensor 130b.

Meanwhile, the controller 300 may control driving of the first UV LED 140a to be suspended according to the touch motion around the down button 110b. Also, the controller 300 may drive the second UV LED 140b and may control the second UV LED 140b such that the UV sterilization and disinfection is performed in an area around the down button 110b. Therefore, the controller 300 may suspend the disinfection and sterilization operation on the up button 110a according to the touch motion around the down button 110b and may perform the disinfection and sterilization operation on the down button 110b.

As another example, the controller 300 may control the first transmission sensor 120a to emit IR rays according to a touch motion around the up button 110a during the UV sterilization and disinfection in the area around the down button 110b. The controller 300 may recognize that the up button 110a is pressed by receiving the IR rays at the first reception sensor 130a.

Meanwhile, the controller 300 may control driving of the second UV LED 140b to be suspended according to the touch motion around the up button 110a. Also, the controller 300 may drive the first UV LED 140a and may control the second UV LED 140b such that UV sterilization and disinfection is performed in the area around the up button 110a. Therefore, the controller 300 may suspend the disinfection and sterilization operation on the down button 110b and may perform the disinfection and sterilization operation on the up button 110a according to the touch motion around the up button 110a.

Referring to FIG. 2, the internal operation panel 200 may be configured to control a floor on which the elevator arrives and stops and opening and closing of the door of the elevator inside the elevator. Meanwhile, FIG. 5 illustrates a detailed configuration of an elevator operation system according to another embodiment of the present invention. In this regard, the elevator operation system may operably couple to the internal operation panel.

Referring to FIGS. 2 and 5, the controller 300 may be configured to be operable with the internal operation panel 200. The controller 300 may control a sensor and an LED associated with a corresponding button based on a user input on the open button 210a, the close button 210b, and the plurality of buttons 220 corresponding to a floor to be stopped of the internal operation panel 200. In this regard, the open button 210a, the close button 210b, and the plurality of buttons 220 corresponding to the floor to be stopped of the internal operation panel 200 may be arranged in the internal area at a predetermined interval from the plane of the internal operation panel 200.

The controller 300 may be configured to recognize a touch motion around a first button among the open button 210a, the close button 210b, and the plurality of buttons 220 corresponding to the floor to be stopped of the internal operation panel 200. The controller 300 may control a first transmission sensor closest to the first button to emit IR rays according to the touch motion around the first button. Also, the controller 300 may recognize that the first button is pressed by receiving the IR rays at the first reception sensor. In this regard, the first button may be one of the open button 210a, the close button 210b, and the plurality of buttons 220 corresponding to the floor to be stopped. Meanwhile, the first transmission sensor and the first reception sensor may be sensors configured to recognize a touch motion on a corresponding button.

When the first button is recognized as being pressed, the controller 300 may drive the first UV LED and may control the first UV LED such that UV sterilization and disinfection is performed in an area around the first button. Also, when the first button is recognized as being pressed again during the UV sterilization and disinfection by the first UV LED, the controller 300 may suspend driving of the first UV LED.

According to an embodiment of the present invention, the controller 300 may be configured to recognize a button touch motion on a specific floor among buttons respectively corresponding to a plurality of floors. In this regard, the controller 300 may control a second transmission sensor closest to a second button to emit IR rays according to a touch motion around the second button among the plurality of buttons 220 of the internal operation panel. The controller 300 may recognize that the second button is pressed by receiving the IR rays at the second reception sensor. In this regard, the second button may be one of the plurality of buttons 220 corresponding to the floor to be stopped. Meanwhile, the second transmission sensor and the second reception sensor may be sensors configured to recognize a touch motion on one button among the plurality of buttons 220 corresponding to the floor to be stopped.

Meanwhile, when a button touch motion on a specific floor among buttons respectively corresponding to a plurality of floors is recognized, the controller 300 may suspend a disinfection and sterilization operation on a button corresponding to another floor. In this regard, when the second button is recognized as being pressed, the controller 300 may control driving of the first UV LED to be suspended. Also, the controller 300 may drive the second UV LED and may control the second UV LED such that UV sterilization and disinfection is performed in an area around the second button.

Meanwhile, the controller 300 may recognize a touch button motion again while a disinfection and sterilization operation being performed on a button corresponding to a specific floor according to a button touch motion on the specific floor. In this regard, when the button touch motion is recognized again during the disinfection and sterilization operation on the button corresponding to the specific floor, the controller 300 may suspend the disinfection and sterilization operation on the corresponding button in consideration of the intent of the user. When the second button is recognized as being pressed again during the UV sterilization and disinfection by the second UV LED, the controller 300 may suspend driving of the second UV LED.

According to another embodiment of the present invention, when a disinfection and sterilization effect by a specific. UV LED is insufficient, disinfection and sterilization by another UV LED may be simultaneously performed. In this regard, the controller 300 may determine whether light intensity of the first UV LED is less than or equal to a threshold during the UV sterilization and disinfection by the first UV LED. If the light intensity of the first UV LED is less than or equal to the threshold, the controller 300 may drive a third UV LED closest to the first UV LED and control UV sterilization and disinfection to be performed in an area around the first button.

According to still another embodiment of the present invention, disinfection and sterilization by at least one UV LED may be simultaneously performed based on a distance between a plurality of buttons and a user touch motion. In this regard, the controller 300 may determine whether light intensity of the first UV LED is less than or equal to a threshold during the UV sterilization and disinfection by the first UV LIED. If the light intensity of the first UV LED is less than or equal to the threshold, the controller 300 may determine whether a touch motion is received around a fourth button within a predetermined distance form the first button. The controller 300 may drive a fourth UV LED corresponding to the fourth button and may control UV sterilization and disinfection to be performed in an area around the first button.

An elevator operation method and an elevator operation system for performing the same according to the present invention are described above. The technical effects according to the present invention may be provided as follows.

According to the present invention, it is possible to provide an elevator operation system that may sterilize and disinfect a corresponding button every time a user touches an elevator button.

Also, according to the present invention, it is possible to provide a button disinfection method that may sterilize and disinfect a corresponding button every time a user touches an elevator button and an elevator operation system for performing the same.

Also, according to the present invention, it is possible to provide a button disinfection method that may sterilize and disinfect a corresponding button by reflecting the intent of a user every time each button provided to an external operation panel of an elevator is touched and an elevator operation system for performing the same.

Also, according to the present invention, it is possible to provide a button disinfection method that may sterilize and disinfect a corresponding button by reflecting the intent of a user every time each button provided to an internal operation panel of an elevator is touched and an elevator operation system for performing the same.

Also, according to the present invention, it is possible to provide a button disinfection method that may sterilize and disinfect a corresponding button in consideration of the intent of a user and a sterilization and disinfection capability associated with each button every time each button provided to an internal operation panel or an external operation panel of an elevator is touched and an elevator operation system for performing the same.

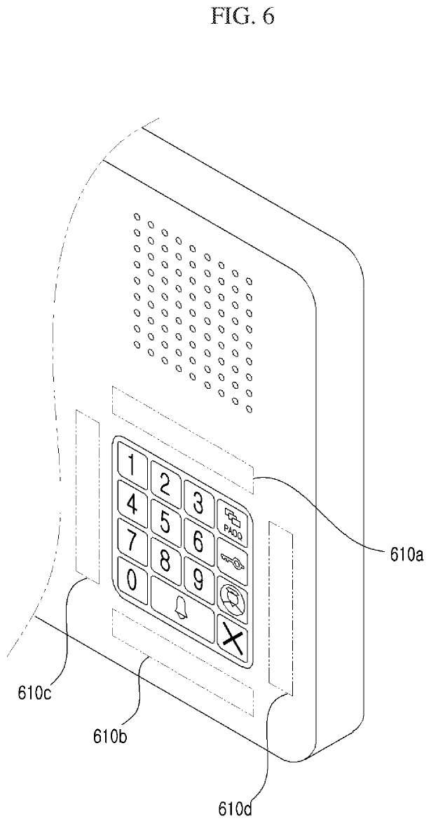

Meanwhile, referring to FIGS. 1 to 5, an elevator button disinfection and sterilization method and an elevator operation system for performing the same disclosed herein may apply to an entrance door of a house or a common front door. In this regard, FIG. 6 illustrates an external operation panel for controlling a common front door according to another embodiment of the present invention. Referring to FIG. 6, the button disinfection and sterilization method disclosed in FIGS. 1 to 5 may apply to the external operation panel for controlling the common front door.

In this regard, a common front door with a public key panel ay be installed at the entrance of an apartment, a multi-family house, a villa, an office hotel, and the like. This public key panel has an issue in that contaminants may be smeared on each button since many users use the public key panel to press buttons even a day. To solve this issue, the present invention may automatically sterilize a pressed button by mounting sterilization lamps 610a, 610b), 610c, and 610d next to buttons of the public key panel and may solve the issue, such as contaminants, bacteria, and viruses. Meanwhile, a location of a sterilization lamp is not limited to an illustrated location and may be installed for each button as shown in FIGS. 2 and 3.

The external operation panel for controlling the common front door includes a plurality of buttons corresponding to different numbers. Therefore, in addition to the button disinfection and sterilization method in the external operation panel disclosed in FIGS. 1 to 5, the button disinfection and sterilization method in the internal operation panel may apply.

The features and effect of the present invention will become apparent through the following detailed description by referring to the accompanying drawings. Therefore, the technical spirit of the present invention may be easily implemented by one of ordinary skill in the art to which the present invention pertains.

The features and effect of the present invention will become apparent through the following detailed description by referring to the accompanying drawings. Therefore, the technical spirit of the present invention may be easily implemented by one of ordinary skill in the art to which the present invention pertains.

Since various modifications may be made to the present invention and various embodiments may be implemented, specific embodiments will be illustrated in the drawings and described in detail in the detailed description. However, the present invention is not construed as being limited to specific implementation and it should be understood to include all modification, equivalents, and substitutes included in the spirit and technical scope of the present invention.

According to software implementation, not only procedures and functions described herein but also design and parameter optimizations related to each of components may be implemented as a separate software module. A software code may be implemented as a software application written in an appropriate program language. The software code may be stored in a memory and may be executed by a controller or a processor.

What is claimed is:

1. An elevator operation system comprising:
an external operation panel configured to control stopping of an elevator and opening and closing of a door of the elevator from the outside of the elevator; and
a controller configured to be operable with the external operation panel and to control a sensor and a light emitting diode (LED) associated with an up button and a down button of the external operation panel based on a user input on the up button and the down button of the external operation panel,
wherein the up button and the down button are provided in an internal area at a predetermined interval from the plane of the external operation panel, and
the controller is configured to,
recognize that the up button is pressed by emitting infrared (IR) rays from a first transmission sensor according to a touch motion around the up button and by receiving the IR rays at a first reception sensor, and
when the up button is recognized as being pressed, drive a first ultraviolet (UV) LED and control the first UV LED such that UV sterilization and disinfection is performed in an area around the up button.

2. The elevator operation system of claim 1, wherein the controller is configured to,
recognize that the down button is pressed by emitting IR rays from a second transmission sensor according to a touch motion around the down button and by receiving the IR rays at a second reception sensor, and
when the down button is recognized as being pressed, drive a second UV LED and control the second UV LED such that UV sterilization and disinfection is performed in an area around the down button.

3. The elevator operation system of claim 2, wherein controller is configured to,
when the up button is recognized as being pressed again during the UV sterilization and disinfection by the first UV LED, suspend driving of the first UV LED, and
when the down button is recognized as being pressed again during the UV sterilization and disinfection by the second UV LED, suspend driving of the second UV LED.

4. The elevator operation system of claim 1, wherein the controller is configured to,
recognize that the down button is pressed by emitting IR rays from a second transmission sensor according to a touch motion around the down button during the UV sterilization and disinfection in the area around the up button, and by receiving the IR rays at a second reception sensor,
suspend driving of the first UV LED according to a touch motion around the down button, drive a second UV LED, and control the second UV LED such that UV sterilization and disinfection is performed in an area around the down button.

5. The elevator operation system of claim 1, further comprising:
an internal operation panel configured to control a floor on which the elevator arrives and stops and opening and closing of the door of the elevator inside the elevator,
wherein the controller is configured to be operable with the internal operation panel and control a sensor and an LED associated with a corresponding button based on a user input on an open button, a close button, and a plurality of buttons corresponding to the floor to be stopped of the internal operation panel.

6. The elevator operation system of claim 5, wherein the open button, the close button, and the plurality of buttons corresponding to the floor to be stopped of the internal operation panel are arranged in an internal area at a predetermined interval from the plane of the internal operation panel, and
the controller is configured to,
recognize that a first button is pressed by emitting IR rays from a first transmission sensor closest to the first button according to a touch motion around the first button among the open button, the close button, and the plurality of buttons corresponding to the floor to be stopped of the internal operation panel and by receiving the IR rays at the first reception sensor, and
when the first button is recognized as being pressed, drive a first UV LED and control the first UV LED such that UV sterilization and disinfection is performed in an area around the first button.

7. The elevator operation system of claim 6, wherein the controller is configured to, when the first button is recognized as being pressed again during the UV sterilization and disinfection by the first UV LED, suspend driving of the first UV LED.

8. The elevator operation system of claim 7, wherein the controller is configured to recognize that a second button is pressed by emitting IR rays from a second transmission sensor closest to the second button according to a touch motion around the second button among the plurality of buttons of the internal operation panel and by receiving the IR rays at the second reception sensor, and
when the second button is recognized as being pressed again, suspend driving of the first UV LED, drive a second UV LED, and control the second UV LED such that UV sterilization and disinfection is performed in an area around the second button.

9. The elevator operation system of claim 8, wherein the controller is configured to, when the second button is recognized as being pressed again during the UV sterilization and disinfection by the second UV LED, suspend driving of the second UV LED.

10. The elevator operation system of claim 6, wherein the controller is configured to, if light intensity of the first UV LED is less than or equal to a threshold during the UV sterilization and disinfection by the first UV LED, drive a third UV LED closest to the first UV LED and control UV sterilization and disinfection to be performed in the area around the first button.

11. The elevator operation system of claim 6, wherein the controller is configured to, if light intensity of the first UV LED is less than or equal to a threshold during the UV sterilization and disinfection by the first UV LED and a touch motion is received around a fourth button within a predetermined distance from the first button, drive a fourth UV LED corresponding to the fourth button and control UV sterilization and disinfection to be performed in the area around the first button.
